(19) United States
(12) Patent Application Publication
(10) Pub. No.: US 2013/0237779 A1
(43) Pub. Date: Sep. 12, 2013

(54) Systems and Methods to Mitigate the Effects of Skin Moisture on a Percutaneous Infrared Signal

(71) Applicants: IVWATCH, LLC, Williamsburg, VA (US); GARY P. WARREN, Williamsburg, VA (US); MATTHEW S. ALLEY, Williamsburg, VA (US)

(72) Inventors: Gary P. Warren, Williamsburg, VA (US); Matthew S. Alley, Sandston, VA (US)

(73) Assignees: Gary P. Warren, Williamsburg, VA (US); ivWatch, LLC, Williamsburg, VA (US); Matthew S. Alley, Williamsburg, VA (US)

(21) Appl. No.: 13/792,096

(22) Filed: Mar. 10, 2013

Related U.S. Application Data
(60) Provisional application No. 61/681,231, filed on Aug. 9, 2012.
(60) Provisional application No. 61/609,865, filed on Mar. 12, 2012.

(51) Int. Cl. A61M 5/168

(52) U.S. Cl. CPC A61M 5/16836
USPC 600/306; 600/407; 600/473

(57) ABSTRACT

A system and method include a sensor overlying a target area of epidermis to aid in diagnosing subcutaneous fluid leakage. The system includes an antiperspirant and an adhesive coupling the sensor and epidermis. The antiperspirant mitigates moisture content variation of the target area, which the inventors discovered is a source of unreliable indications that an infiltration or extravasation event has occurred.

### CROSS-REFERENCES TO RELATED APPLICATIONS

This application claims the priority of U.S. Provisional Application No. 61/681,231, filed 9 Aug. 2012, and also claims the priority of U.S. Provisional Application No. 61/609,865, filed 12 Mar. 2012, each of which are hereby incorporated by reference in their entirety.

### STATEMENT REGARDING FEDERALLY SPONSORED RESEARCH OR DEVELOPMENT

Not Applicable

### BACKGROUND OF THE INVENTION

FIG. 6 shows a typical arrangement for intravascular infusion. As the terminology is used herein, “intravascular” preferably refers to being situated in, occurring in, or being administered by entry into a blood vessel, thus “intravascular infusion” preferably refers to introducing a fluid or infusate into a blood vessel. Intravascular infusion accordingly encompasses both intravenous infusion (administering a fluid into a vein) and intra-arterial infusion (administering a fluid into an artery).

A cannula 20 is typically used for administering fluid via a subcutaneous blood vessel V. (See FIG. 1.) Typically, cannula 20 is inserted through epidermis E at a cannulation site S and punctures, for example, the cephalic vein, basilica vein, median cubital vein, or any suitable vein for an intravenous infusion. Similarly, any suitable artery may be used for an intra-arterial infusion.

Cannula 20 typically is in fluid communication with a fluid source 22. Typically, cannula 20 includes an extracorporeal connector, e.g., a hub 20a, and a transcutaneous sleeve 20b. Fluid source 22 typically includes one or more sterile containers that hold the fluid(s) to be administered. Examples of typical sterile containers include plastic bags, glass bottles or plastic bottles.

An administration set 30 typically provides a sterile conduit for fluid to flow from fluid source 22 to cannula 20. Typically, administration set 30 includes tubing 32, a drip chamber 34, a flow control device 36, and a cannula connector 38. Tubing 32 is typically made of polypropylene, nylon, or another flexible, strong and inert material. Drip chamber 34 typically permits the fluid to flow one drop at a time for reducing air bubbles in the flow. Tubing 32 and drip chamber 34 are typically transparent or translucent to provide a visual indication of the flow. Typically, flow control device 36 is positioned upstream from drip chamber 34 for controlling fluid flow in tubing 34. Roller clamps and Dial-A-Flo®, manufactured by Hospira, Inc. (Lake Forest, Ill., USA), are examples of typical flow control devices. Typically, cannula connector 38 and hub 20a provide a leak-proof coupling through which the fluid may flow. Luer-Lok™, manufactured by Becton, Dickinson and Company (Franklin Lakes, N.J., USA), is an example of a typical leak-proof coupling.

Administration set 30 may also include at least one of a clamp 40, an injection port 42, a filter 44, or other devices. Typically, clamp 40 pinches tubing 32 to cut-off fluid flow. Injection port 42 typically provides an access port for administering medicine or another fluid via cannula 20. Filter 44 typically purifies and/or treats the fluid flowing through administration set 30. For example, filter 44 may strain contaminants from the fluid.

An infusion pump 50 may be coupled with administration set 30 for controlling the quantity or the rate of fluid flow to cannula 20. The Alaris® System manufactured by CareFusion Corporation (San Diego, Calif., USA) and Flo-Gard® Volumetric Infusion Pumps manufactured by Baxter International Inc. (Deerfield, Ill., USA) are examples of typical infusion pumps.

Intravenous infusion or therapy typically uses a fluid (e.g., infusate, whole blood, or blood product) to correct an electrolyte imbalance, to deliver a medication, or to elevate a fluid level. Typical infusates predominately consist of sterile water with electrolytes (e.g., sodium, potassium, or chloride), calories (e.g., dextrose or total parenteral nutrition), or medications (e.g., anti-infectives, anticonvulsants, antihyperuricemic agents, cardiovascular agents, central nervous system agents, chemotherapy drugs, coagulation modifiers, gastrointestinal agents, or respiratory agents). Examples of medications that are typically administered during intravenous therapy include acyclovir, allopurinol, amikacin, aminophylline, amiodarone, amphotericin B, ampicillin, carboplatin, cefazolin, cefotaxime, cefuroxime, ciprofloxacin, cisplatin, clindamycin, cyclophosphamide, diazepam, docetaxel, dopamine, doxorubicin, doxycycline, erythromycin, etoposide, fentanyl, fluorouracil, furosemide, ganciclovir, gemcitabine, gentamicin, heparin, imipenem, irinotecan, lorazepam, magnesium sulfate, meropenem, methotrexate, methylprednisolone, midazolam, morphine, nafcillin, ondansetron, paclitaxel, pentamidine, phenobarbital, phenytoin, piperacillin, promethazine, sodium bicarbonate, ticarcillin, tobramycin, topotecan, vancomycin, vinblastine and vincristine. Transfusions and other processes for donating and receiving whole blood or blood products (e.g., albumin and immunoglobulin) also typically use intravenous infusion.

Unintended infusing typically occurs when fluid from cannula 20 escapes from its intended vein/artery. Typically, unintended infusing causes an abnormal amount of the fluid to diffuse or accumulate in perivascular tissue and may occur, for example, when (i) cannula 20 causes a brittle vein/artery to rupture; (ii) cannula 20 improperly punctures the vein/artery; (iii) cannula 20 is improperly sized; or (iv) infusion pump 50 administers fluid at an excessive flow rate. As the terminology is used herein, “perivascular tissue” preferably refers to the cells and/or interstitial compartments that are in the vicinity of a blood vessel and may become unintentionally infused with fluid from cannula 20. Unintended infusing of a non-vesicant fluid is typically referred to as “infiltration,” whereas unintended infusing of a vesicant fluid is typically referred to as “extravasation.”

The symptoms of infiltration or extravasation typically include blanching or discoloration of the epidermis E, edema, pain, or numbness. The consequences of infiltration or extravasation typically include skin reactions such as blisters, nerve compression, compartment syndrome, or necrosis. Typical treatment for infiltration or extravasation includes applying warm compresses, administering hyaluronidase, phentolamine, sodium thiosulfate or dexrazoxane, fasciotomy, or amputation.

### BRIEF SUMMARY OF THE INVENTION

Embodiments according to the present invention include a system for aiding in diagnosing subcutaneous fluid leakage with a sensor that overlies a target area of epidermis. The system includes an antiseptic agent, an antiperspirant, and a foundation configured to couple the sensor and epidermis. The antiseptic agent is configured to clean the target area. The antiperspirant is configured to minimize moisture content variation of the target area. The foundation includes an adhesive.

Other embodiments according to the present invention include a system for monitoring an intravascular infusion. The system includes a sensor and antiperspirant. The sensor is configured to emit a first percutaneous electromagnetic signal and to receive a second percutaneous electromagnetic signal. The second percutaneous electromagnetic signal includes at least one of a reflection, scattering and diffusion of the first percutaneous electromagnetic signal. The antiperspirant is configured to be disposed at a target area of epidermis, and the first and second percutaneous electromagnetic signals pass through the target area.

Other embodiments according to the present invention include a system for monitoring an anatomical property of a body that includes an epidermis. The system includes a sensor and an antiperspirant. The sensor is configured to overlie a target area of the epidermis and to receive a first signal regarding the anatomical property. The antiperspirant is configured to minimize moisture content variation of the target area of the epidermis.

Other embodiments according to the present invention include a system for monitoring a subcutaneous anatomical property of a body that includes an epidermis. The system includes first and second sensors. The first sensor is configured to overlie a target area of the epidermis and to receive a first signal regarding the subcutaneous anatomical property. The second sensor is configured to receive a second signal regarding moisture content of the target area of the epidermis.

Other embodiments according to the present invention include a method of sensing fluid in perivascular tissue. The method includes applying an antiperspirant to a target area of epidermis and coupling a sensor to the epidermis. The sensor is configured to emit and detect near-infrared signals through the target area.

Other embodiments according to the present invention include a method of monitoring an anatomical property of a body including an epidermis. The method includes applying an antiperspirant to a target area of the epidermis and sensing at the target area a signal regarding the anatomical property.

Other embodiments according to the present invention include a method of monitoring an intravascular infusion. The method includes sensing fluid in perivascular tissue. Sensing the fluid includes detecting a first percutaneous near-infrared signal passing through a target area of epidermis. The method further includes sensing moisture content of the epidermis at the target area, and compensating the first percutaneous near-infrared signal based on the moisture content of the epidermis at the target area.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features, principles, and methods of the invention.

FIG. 1 is a schematic cross-section view illustrating an embodiment of an electromagnetic spectrum sensor according to the present disclosure.

FIGS. 2A-2D illustrate examples of changes in percutaneous transmission of electromagnetic radiation that the inventors discovered are caused by moisture content variation of skin.

FIG. 3 is a flow chart illustrating an embodiment of a method according to the present disclosure for annulling epidermal moisture content variation.

FIG. 4A is a schematic cross-section view illustrating an embodiment of a bi-spectral sensor according to the present disclosure to compensate for epidermal moisture content variation.

FIG. 4B is a schematic illustration of a signal processing system including the bi-spectral sensor shown in FIG. 4A.

FIG. 5A is a schematic cross-section view illustrating an embodiment of a combination sensor according to the present disclosure to compensate for epidermal moisture content variation.

FIG. 5B is a schematic illustration of a signal processing system including the combination sensor shown in FIG. 5A.

FIG. 6 is a schematic view illustrating a typical set-up for infusion administration.

In the figures, the thickness and configuration of components may be exaggerated for clarity. The same reference numerals in different figures represent the same component.

### DETAILED DESCRIPTION OF THE INVENTION

The following description and drawings are illustrative and are not to be construed as limiting. Numerous specific details are described to provide a thorough understanding of the disclosure. However, in certain instances, well-known or conventional details are not described in order to avoid obscuring the description.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various features are described which may be included in some embodiments but not other embodiments.

The terms used in this specification generally have their ordinary meanings in the art, within the context of the disclosure, and in the specific context where each term is used. Certain terms in this specification may be used to provide additional guidance regarding the description of the disclosure. It will be appreciated that a feature may be described more than one-way.

Alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term.

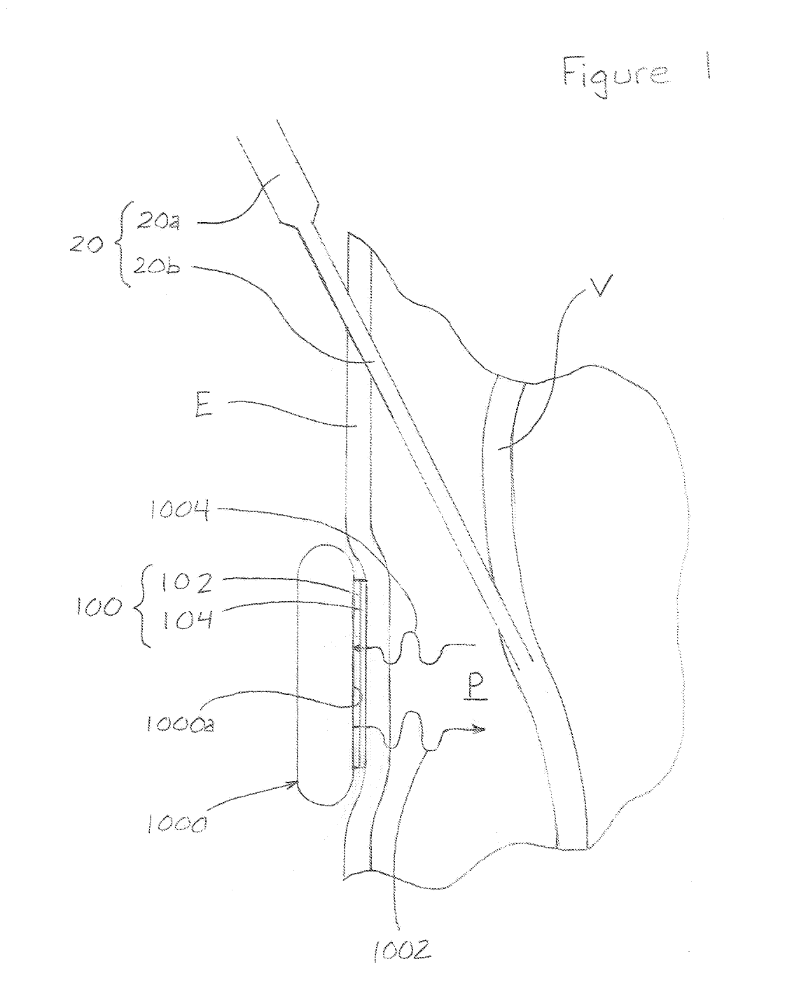

FIG. 1 shows an electromagnetic spectrum sensor 1000 preferably coupled with the epidermis E. Preferably, electromagnetic spectrum sensor 1000 includes an anatomic sensor. As the terminology is used herein, “anatomic” preferably refers to the structure of an Animalia body and an “anatomic sensor” preferably is concerned with sensing a change over time of the structure of the Animalia body. By comparison, a physiological sensor is concerned with sensing the functions and activities of an Animalia body, e.g., pulse, at a point in time.

Electromagnetic spectrum sensor 1000 includes a sensor face 1000a preferably arranged to confront or overlie a target area of the epidermis E for aiding in diagnosing infiltration or extravasation. As the terminology is used herein, “target area” preferably refers to a portion of a patient's epidermis that is generally proximal to where an infusate is being administered and frequently proximal to the cannulation site S. Preferably, electromagnetic radiation 1002 is emitted via sensor face 1000a and received electromagnetic radiation 1004 is received via sensor face 1000a. Emitted electromagnetic radiation 1002 preferably passes through the target area of the epidermis E into perivascular tissue P. Preferably, infiltration or extravasation of the perivascular tissue P by an infusate fluid affects the absorption of emitted electromagnetic radiation 1002. Received electromagnetic radiation 1004 preferably includes at least a portion of emitted electromagnetic radiation 1002 that is reflected, scattered, diffused, or otherwise redirected from the perivascular tissue P and/or infusate fluid, through the target area of the epidermis E, to sensor face 1000a. Accordingly, infiltration or extravasation of the perivascular tissue P with the infusate fluid preferably also affects received electromagnetic radiation 1004. Electromagnetic spectrum sensor 1000 therefore preferably detects changes in received electromagnetic radiation 1004 that correspond with anatomic changes over time due to accumulation of infusate fluid in the perivascular tissue P. Acute limb compartment syndrome is an example of such an anatomic change.

Emitted and received electromagnetic radiations 1002 and 1004 preferably are in the near-infrared portion of the electromagnetic spectrum. As the terminology is used herein, “near infrared” preferably refers to electromagnetic radiation having wavelengths between approximately 750 nanometers and approximately 1,400 nanometers. These wavelengths correspond to a frequency range of approximately 400 terahertz to approximately 215 terahertz. Preferably, emitted and received electromagnetic radiations 1002 and 1004 are tuned to a common peak wavelength. According to one embodiment, emitted and received electromagnetic radiations 1002 and 1004 each have a peak centered about a single wavelength, e.g., approximately 970 nanometers (approximately 309 terahertz). According to other embodiments, emitted electromagnetic radiation 1002 includes a set of wavelengths in a band between a relatively low wavelength and a relatively high wavelength, and received electromagnetic radiation 1004 encompasses at least the band between the relatively low and high wavelengths. According to still other embodiments, received electromagnetic radiation 1004 is tuned to a set of wavelengths in a band between a relatively low wavelength and a relatively high wavelength, and emitted electromagnetic radiation 1002 encompasses at least the band between the relatively low and high wavelengths.

Electromagnetic spectrum sensor 1000 preferably is positioned in close proximity to the epidermis E. As the terminology is used herein, “close proximity” of electromagnetic spectrum sensor 1000 and the epidermis E preferably refers to a relative arrangement that substantially eliminates gaps between sensor face 1000a and the epidermis E. According to one embodiment, sensor face 1000a preferably contiguously engages the epidermis E. According to other embodiments, a foundation 100 preferably is disposed between electromagnetic spectrum sensor 1000 and the epidermis E. Preferably, sensor face 1000a contiguously engages foundation 100 and foundation 100 contiguously engages the epidermis E.

Foundation 100 preferably includes a panel 102 and/or adhesive 104 coupled with respect to sensor face 1000a. Preferably, panel 102 separates electromagnetic spectrum sensor 1000 from the epidermis E. Panel 102 preferably includes Tegaderm™, manufactured by 3M (St. Paul, Minn., USA), REACTIC™, manufactured by Smith & Nephew (London, UK), or another transparent or translucent polymer film that is substantially impervious to solids, liquids, microorganisms and/or viruses. Preferably, panel 102 is biocompatible and generally transparent with respect to emitted and received electromagnetic radiations 1002 and 1004. As the terminology is used herein, “biocompatible” preferably refers to compliance with Standard 10993 promulgated by the International Organization for Standardization (ISO 10993) and/or Class VI promulgated by The United States Pharmacopeial Convention (USP Class VI). Other regulatory entities, e.g., National Institute of Standards and Technology, may also promulgate standards that may additionally or alternatively be applicable regarding biocompatibility.

Adhesive 104 preferably bonds at least one of electromagnetic spectrum sensor 1000 and panel 102 to the epidermis E. Adhesive 104 preferably includes an acrylic adhesive or another medical grade, biocompatible adhesive. Preferably, adhesive 104 minimally affects the transmission of emitted and received electromagnetic radiations 1002 and 1004. According to one embodiment, adhesive 104 preferably is omitted where emitted and received electromagnetic radiations 1002 and 1004 penetrate foundation 100.

The inventors discovered a problem regarding percutaneous electromagnetic radiation measurements and inaccurate indications of infiltration/extravasation events. In particular, a problem that the inventors discovered is that some changes in the amount of received electromagnetic radiation 1004 are unrelated to the occurrence of infiltration or extravasation. The inventors discovered that these changes generally occur during an approximately 60-minute period of time that begins with positioning electromagnetic spectrum sensor 1000 in close proximity to the epidermis E. The inventors further discovered that these changes predominately include a drop in the amount of received electromagnetic radiation 1004 that occurs within an approximately 35-minute period of time. The inventors also discovered that these changes occur frequently but inconsistently in a statistically significant patient population. In particular, the inventors discovered that these changes occur in approximately 65% to approximately 85% of patient populations. Thus, the inventors discovered, inter alio, that there is a problem because some changes in the amount of received electromagnetic radiation 1004 do not correlate with occurrences of infiltration/extravasation events.

The inventors also discovered that a source of the problem is moisture content variations of the epidermis E due to, for example, secretion or evaporation of sweat. In particular, the inventors discovered that the source of some changes in the amount of received electromagnetic radiation 1004 is epidermal moisture at least partially mimicking the electromagnetic radiation absorption of an infusate fluid. Thus, the inventors discovered, inter alio, that moisture content variation in the epidermis E affects the amount of received electromagnetic radiation 1004.

The inventors further discovered that moisture content variation of the epidermis E is a source of unreliable measurements by epidermal sensors. As the terminology is used herein, “epidermal sensors” preferably refer to (i) sensors that measure thermal properties, e.g., temperature or heat flux, of the epidermis E; (ii) sensors that measure electrical properties, e.g., resistance or impedance, of the epidermis E; (iii) sensors that measure transmission and/or reflectance of electromagnetic radiation, e.g., visible light or infrared radiation, with respect to the epidermis E or perivascular tissue P; or (iv) sensors that measure other properties or quantities of/through the epidermis E. According to one embodiment, unreliable measurements by epidermal sensors may result in inaccurate indications that an infiltration/extravasation event has occurred. The inventors also discovered that epidermal sensor measurements are affected during periods of time that generally coincide with the moisture content of the epidermis E achieving equilibrium. As the terminology is used herein, “equilibrium” of the moisture content of the epidermis E preferably refers to a generally steady-state overall in the production, transfer and loss of moisture content by/from the epidermis E. The inventors further discovered that the period of time for achieving equilibrium predominantly begins shortly after positioning epidermal sensors in close proximity to the epidermis E; however, other periods of time for achieving equilibrium may begin some time later while the epidermal sensor is still positioned in close proximity to the epidermis E. The inventors additionally discovered that the period of time for achieving equilibrium is generally finite, e.g., equilibrium of the moisture content of the epidermis E is generally achieved in approximately 60 minutes or less and frequently in approximately 35 minutes or less. Thus, the inventors discovered, inter alio, that moisture content variation in the epidermis E is a source of unreliable measurements by epidermal sensors.

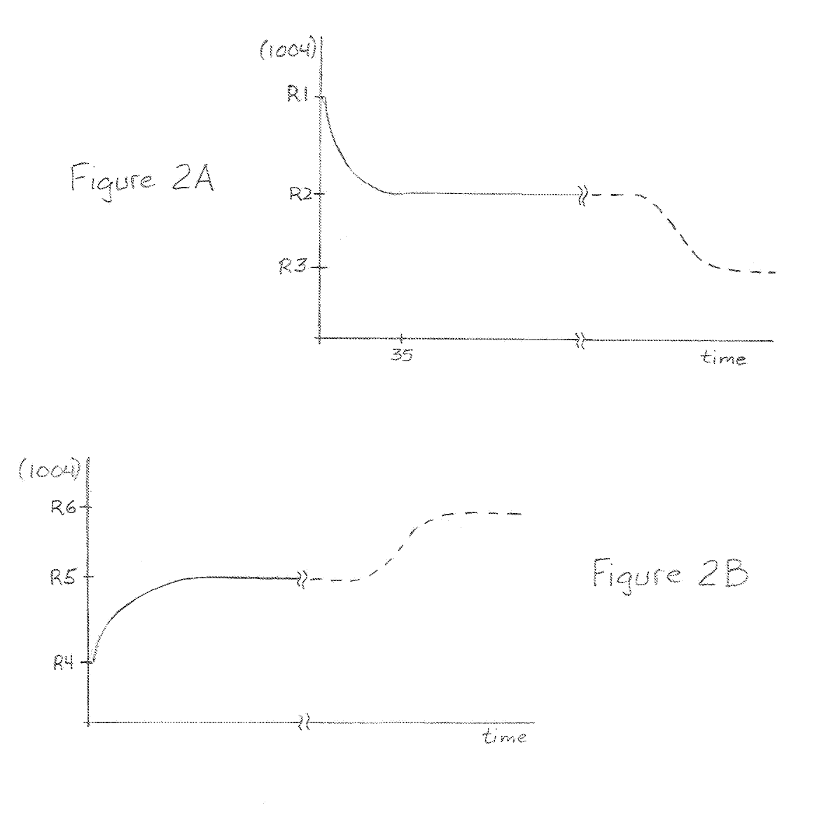

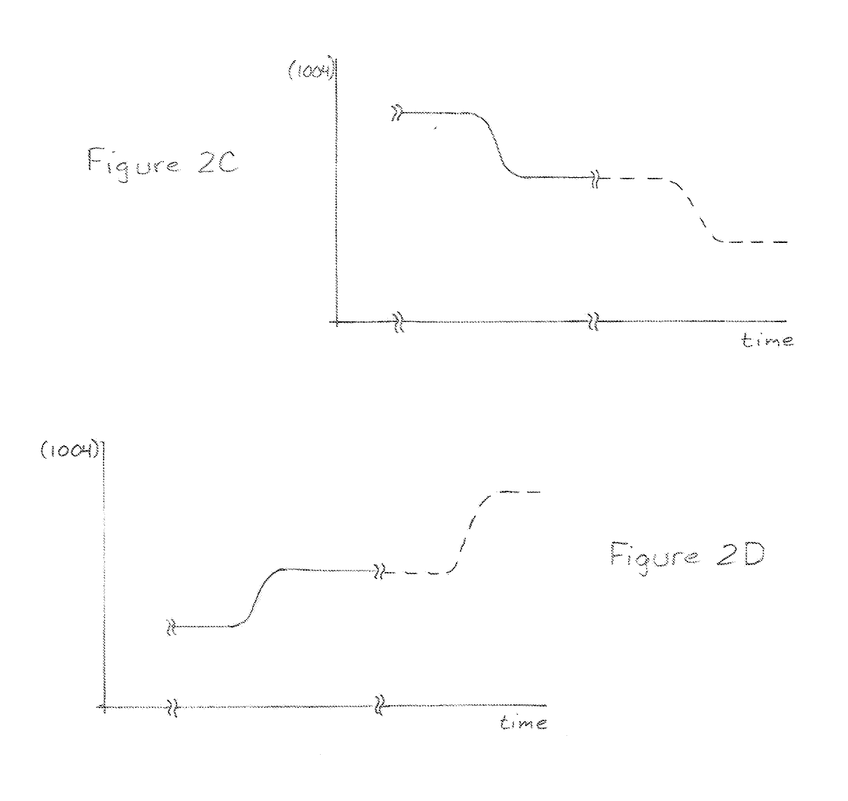

FIGS. 2A-2D illustrate examples of unreliable measurements by epidermal sensors due to moisture content variations of the epidermis E. FIG. 2A illustrates a first example in which there is a first relatively high amount R1 of received electromagnetic radiation 1004 approximately when electromagnetic spectrum sensor 1000 is initially coupled to the epidermis E. The inventors discovered that the amount of received electromagnetic radiation 1004 begins to fall thereafter, e.g., during an approximately 35-minute period of time, and that the fall corresponds to an increase in the moisture content of the epidermis E. Increasing moisture content from a generally depressed level may be due to, for example, (i) patient anxiety; or (ii) interference with normal perspiration and evaporation processes because electromagnetic spectrum sensor 1000 is positioned in close proximity to the epidermis E. Heightened levels of epidermal moisture predominantly correspond to more absorption of emitted electromagnetic radiation 1002. Accordingly, an increase in the moisture content of the epidermis E generally causes a fall in the amount of received electromagnetic radiation 1004 because there is more absorption of emitted electromagnetic radiation 1002. The inventors further discovered that the fall in received electromagnetic radiation 1004 generally continues until the moisture content of the epidermis E achieves equilibrium; whereupon a first relatively low amount R2 of received electromagnetic radiation 1004 may remain generally consistent until and unless an infiltration/extravasation event occurs as shown with the broken line in FIG. 2A. Predominately, emitted electromagnetic radiation 1002 is also absorbed during the infiltration/extravasation event as fluid, e.g., an infusate or blood, accumulates in the perivascular tissue P. Accordingly, there is a second relatively low amount R3 of received electromagnetic radiation 1004 that is caused by fluid accumulation in the perivascular tissue P rather than by moisture content variation in the epidermis E, which was the cause of the first relatively low amount R2 of received electromagnetic radiation 1004. Thus, the inventors discovered, inter alio, that moisture content variation of the epidermis E may be mistaken for an infiltration/extravasation event because a first fall (R1 to R2) in the amount of received electromagnetic radiation 1004 when the moisture content of the epidermis E is achieving equilibrium may be similar to a second fall (R2 to R3) in the amount of received electromagnetic radiation 1004 when an infiltration/extravasation event is occurring.

FIG. 2B illustrates a second example in which there is a first relatively low amount R4 of received electromagnetic radiation 1004 approximately when electromagnetic spectrum sensor 1000 is initially coupled to the epidermis E. The inventors discovered that the amount of received electromagnetic radiation 1004 begins to rise thereafter and that the rise corresponds to a decrease in the moisture content of the epidermis E. Decreasing moisture content from a generally heightened level may be due to, for example, (i) environmental factors such as cold, dry air; (ii) surgery; (iii) bathing; or (iv) interference with normal perspiration and evaporation processes because electromagnetic spectrum sensor 1000 is positioned in close proximity to the epidermis E. Depressed levels of epidermal moisture predominantly correspond to less absorption of emitted electromagnetic radiation 1002. Accordingly, a decrease in the moisture content of the epidermis E generally causes a rise in the amount of received electromagnetic radiation 1004 because there is less absorption of emitted electromagnetic radiation 1002. The inventors further discovered that the rise in received electromagnetic radiation 1004 generally continues until the moisture content of the epidermis E achieves equilibrium; whereupon a first relatively high amount R5 of received electromagnetic radiation 1004 may remain generally consistent until and unless an infiltration/extravasation event occurs as shown with the broken line in FIG. 2B. During the infiltration/extravasation event, a second relatively high amount R6 of received electromagnetic radiation 1004 may be associated with reduced electromagnetic radiation absorption when fluid accumulates in the perivascular tissue P rather than moisture content variation in the epidermis E, which was the cause of the first relatively high amount R5 of received electromagnetic radiation 1004. It is believed that reduced absorption of electromagnetic radiation may be associated with certain wavelengths and/or the electromagnetic spectral signature of certain infusate fluids. As the terminology is used herein, “spectral signature” preferably refers to a pattern of reflected and absorbed electromagnetic wavelengths that particularly corresponds to and therefore identifies a material. Thus, the inventors discovered, inter alio, that moisture content variation of the epidermis E may be mistaken for an infiltration/extravasation event because a first rise (R4 to R5) in the amount of received electromagnetic radiation 1004 when the moisture content of the epidermis E is achieving equilibrium may be similar to a second rise (R5 to R6) in the amount of received electromagnetic radiation 1004 when an infiltration/extravasation event is occurring.

FIGS. 2C and 2D illustrate additional examples in which a change in received electromagnetic radiation 1004 due to moisture content variations of the epidermis E occurs some time after coupling electromagnetic spectrum sensor 1000 to the epidermis E. The inventors discovered that changes in received electromagnetic radiation 1004 may correspond to increasing (FIG. 2C) or decreasing (FIG. 2D) moisture content of the epidermis E that occurs some time after an initial period of time for achieving equilibrium. Thus, the inventors further discovered, inter alio, that a moisture content variation of the epidermis E that occurs some time after equilibrium is initially achieved may be mistaken for an infiltration/extravasation event. The examples in FIGS. 2A-2D therefore illustrate that measurements by epidermal sensors typically may be unreliable because moisture content variations of the epidermis E may be mistaken for infiltration/extravasation events.

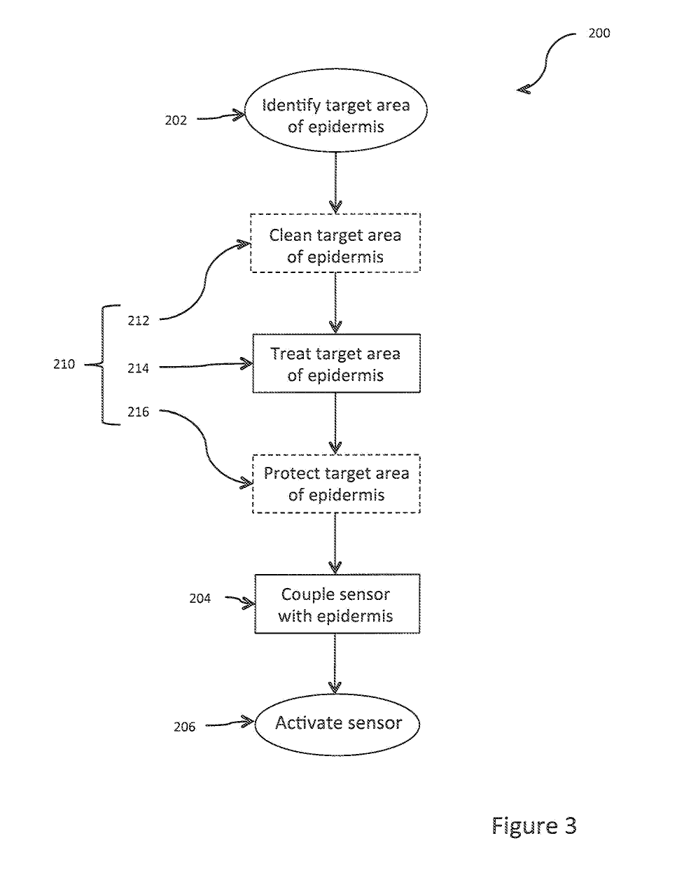

FIG. 3 illustrates an embodiment of a method 200 to substantially achieve equilibrium of the epidermis E by annulling the effects of epidermal moisture content variation. As the terminology is used herein, “annulling” of epidermal moisture content variation preferably refers to eliminating or substantially assuaging variations in the moisture content of the epidermis E. Method 200 preferably begins when a target area of the epidermis E that is to be overlaid by sensor face 1000a is identified 202. Preferably, method 200 includes preparation 210 of the target area of the epidermis E. According to one embodiment, target area preparation 210 preferably includes target area cleaning 212, target area treating 214, and target area protecting 216. Preferably, target area cleaning 212 includes wiping the target area of the epidermis E with a pad soaked with an antiseptic agent such as a solution of approximately 70% isopropyl alcohol/30% deionized water. Other topical applicators and/or antiseptic cleaning agents may be used for target area cleaning 212. Preferably, target area treating 214 includes applying an antiperspirant to the target area. Examples of antiperspirants preferably include those with aluminum-based active ingredients (e.g., aluminum chloride, aluminum zirconium, aluminum chlorohydrate, or aluminum hydroxybromide) or those with aluminum-free active ingredients (e.g., crystal alum or talc). Applying the antiperspirant to the target area of the epidermis E preferably includes wiping with a pad, rolling with a ball, spreading a solid, dousing a powder, or other suitable application techniques. Preferably, target area protecting 216 includes coating the epidermis with a biocompatible barrier film that, for example, minimizes skin trauma, enhances adhesively coupling electromagnetic spectrum sensor 1000 or foundation 100 with respect to the epidermis E, and/or facilitates subsequent decoupling of electromagnetic spectrum sensor 1000 or foundation 100 with respect to the epidermis E. Examples of barrier films preferably include Cavilon™, manufactured by 3M (St. Paul, Minn., USA), or Skin-Prep™, manufactured by Smith & Nephew (London, UK). Thus, according to one embodiment of method 200, target area preparation 210 preferably includes target area cleaning 212, target area treating 214, and target area protecting 216. According to other embodiments, method 200 preferably includes target area treating 214 but may omit target area cleaning 212 and/or target area protecting 216. Preferably, method 200 also includes coupling 204 electromagnetic spectrum sensor 1000 with the target area of the epidermis E and activating 206 electromagnetic spectrum sensor 1000.

Target area treating 214 with an antiperspirant preferably annuls the source of the problem discovered by the inventors. Typically, antiperspirants have an astringent action that tends to reduce the size of skin pores and therefore halt or substantially reduce the passage of moisture via sweat gland ducts. Halting or substantially reducing the passage of moisture via sweat gland ducts preferably eliminates or substantially minimizes epidermal moisture content variations for achieving equilibrium of the epidermis E. Accordingly, target area treating 214 with an antiperspirant preferably annuls epidermal moisture content variation so that measurements with electromagnetic spectrum sensor 1000 may be relied on, for example, as an aid to diagnosing the occurrence of an infiltration/extravasation event.

Target area treating 214 with an antiperspirant preferably occurs no later than when electromagnetic spectrum sensor 1000 is coupled with the epidermis E. According to one embodiment of method 200, target area treating 214 preferably occurs before coupling electromagnetic spectrum sensor 1000 with the epidermis E. Preferably, target area treating 214 occurs after target area cleaning 212 and before target area protecting 216. Other embodiments according to method 200 preferably combine target area treating 214 with coupling electromagnetic spectrum sensor 1000 to the epidermis E. Preferably, foundation 100 includes a mixture of an antiperspirant and adhesive 104 for substantially concurrent target area treating 214 and electromagnetic spectrum sensor 1000 coupling to the epidermis E. Examples of mixtures for foundation 100 may include 1 to 99.9 weight percent adhesive formulation (e.g., elastomers that cure by hydrosilylation or condensation, pressure sensitive adhesives, or other biocompatible adhesives) and 0.1 to 50 weight percent antiperspirant (e.g., anti-diaphoretic compositions that may include aluminum-based active ingredients or aluminum-free active ingredients). Wax or other ingredients may also be included in mixtures for foundation 100.

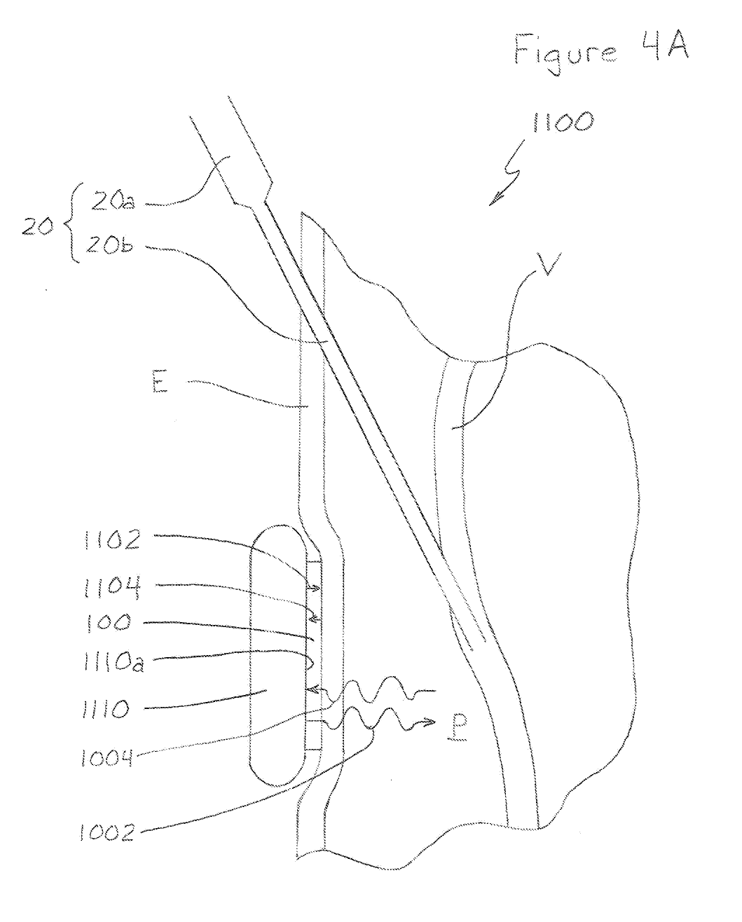

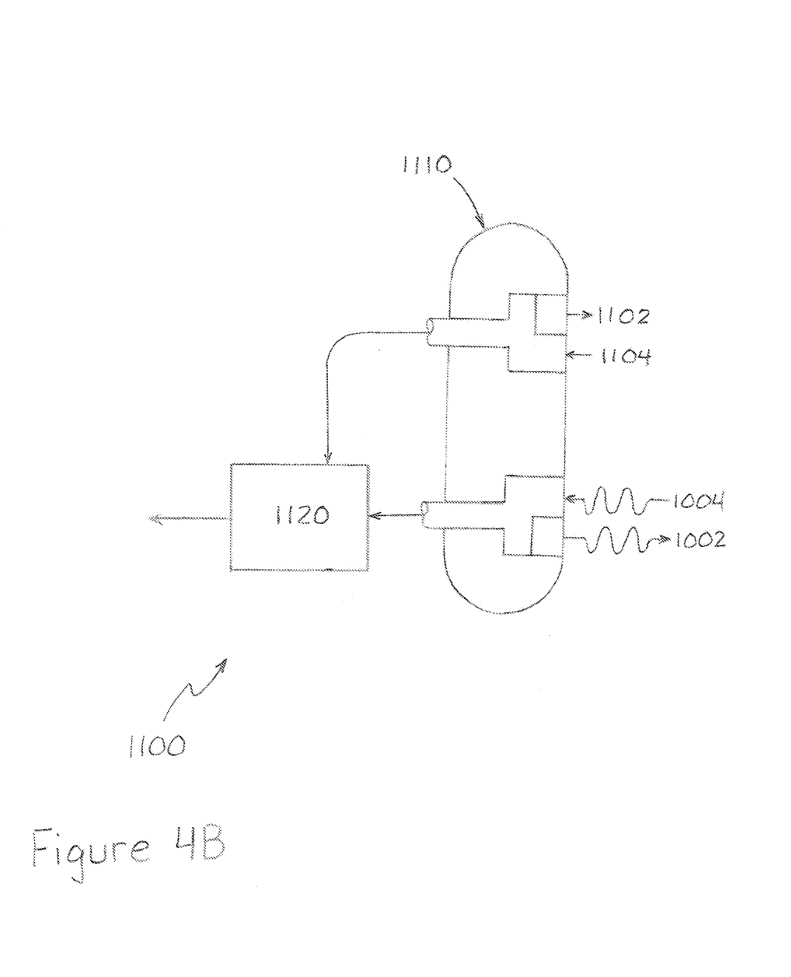

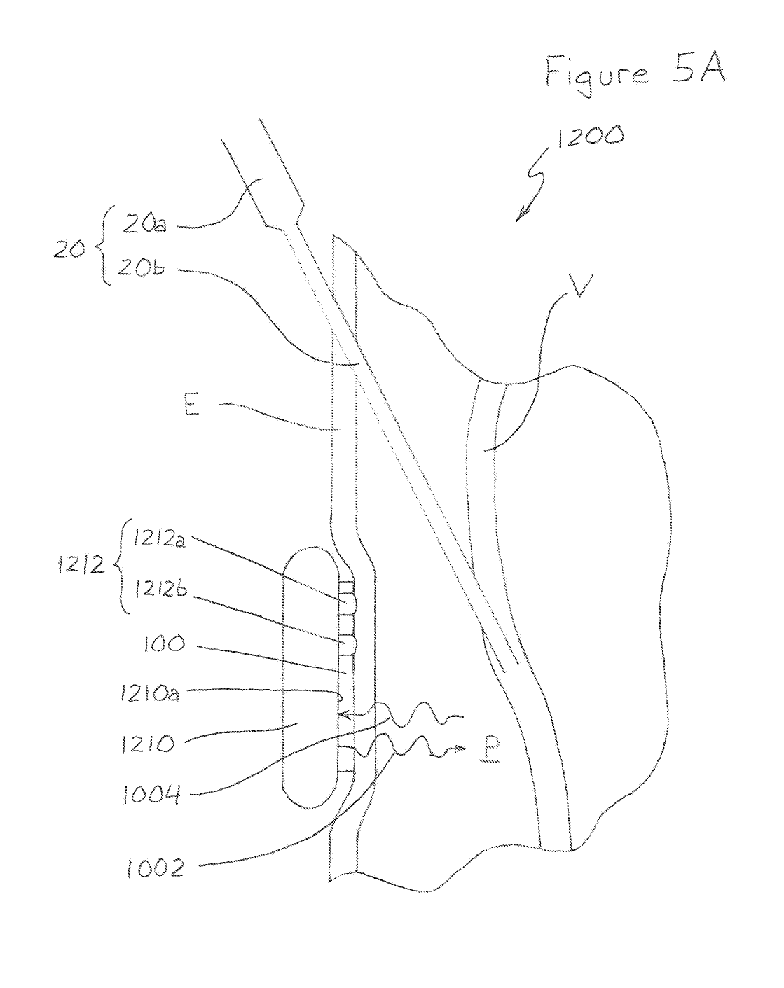

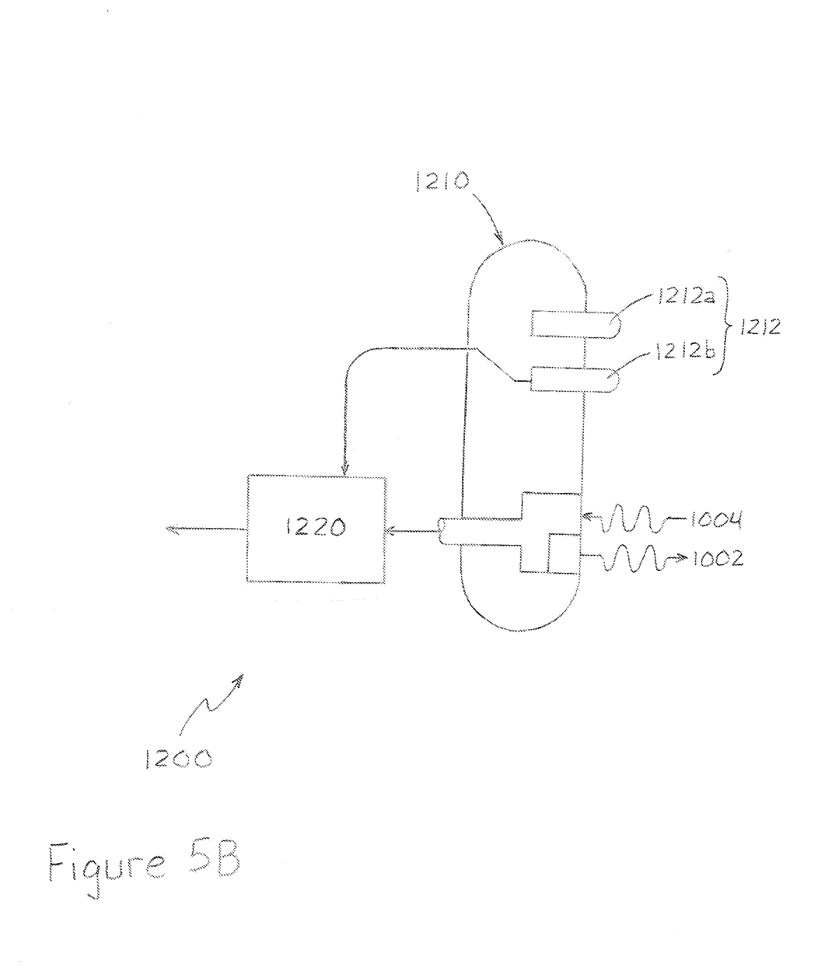

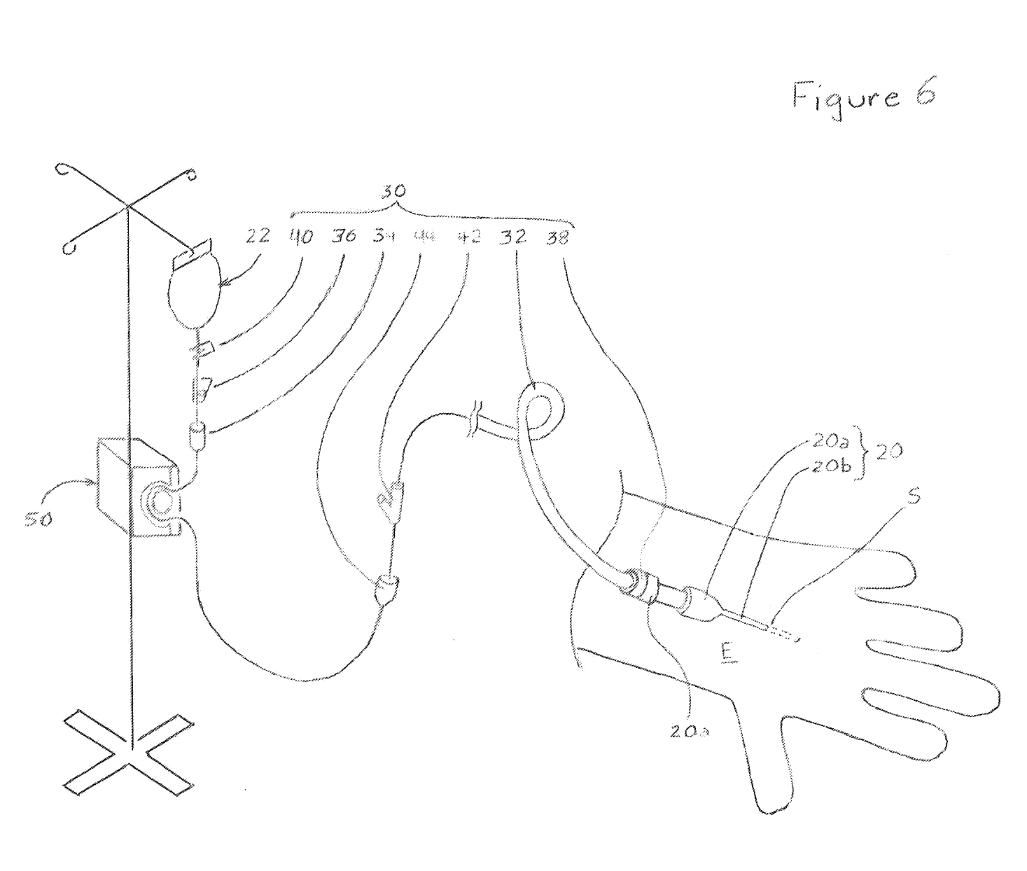

FIGS. 4A-5B illustrate embodiments of a system 1100 for adjusting a percutaneous signal based on a cutaneous signal to compensate for the effects of epidermal moisture content variation. FIG. 4A shows system 1100 including a bi-spectral sensor 1110 coupled with the epidermis E for (i) aiding in diagnosing infiltration or extravasation; and (ii) measuring moisture content variation of the epidermis E. Aiding in diagnosing infiltration or extravasation preferably includes a first electromagnetic radiation 1002 that is emitted via a sensor face 1110a of bi-spectral sensor 1110 and a first electromagnetic radiation 1004 that is received via sensor face 1110a. First emitted and first received electromagnetic radiations 1002 and 1004 of bi-spectral sensor 1110 are generally similar to emitted and received electromagnetic radiations 1002 and 1004 of electromagnetic spectrum sensor 1000 shown in FIG. 1. For example, first emitted and first received electromagnetic radiations 1002 and 1004 preferably are percutaneous signals in the near-infrared portion of the electromagnetic spectrum.

Measuring the moisture content of the epidermis E preferably includes a second electromagnetic radiation 1102 that is emitted via sensor face 1110a and a second electromagnetic radiation 1104 that is received via sensor face 1110a. Second emitted electromagnetic radiation 1102 preferably impinges on the epidermis E and second received electromagnetic radiation 1104 is at least a portion of second emitted electromagnetic radiation 1102 that is reflected, scattered, diffused, or otherwise redirected from the epidermis E to sensor face 1110a. Preferably, second emitted and second received electromagnetic radiations 1102 and 1104 are cutaneous signals and the magnitude of second received electromagnetic radiation 1104 correlates with the moisture content of the epidermis E.

System 1100 preferably uses different portions of the electromagnetic spectrum for aiding in diagnosing infiltration/extravasation events and for measuring moisture content variation of the epidermis E. Preferably, second emitted and second received electromagnetic radiations 1102 and 1104 are in the visible light portion of the electromagnetic spectrum. As the terminology is used herein, “visible light” preferably refers to energy in the visible portion of the electromagnetic spectrum, for example, wavelengths between approximately 390 nanometers and approximately 750 nanometers. These wavelengths correspond to a frequency range of approximately 770 terahertz to approximately 400 terahertz. Preferably, second emitted and second received electromagnetic radiations 1102 and 1104 are tuned to a common peak wavelength. According to one embodiment, second emitted and second received electromagnetic radiations 1102 and 1104 preferably have a peak centered about a single wavelength, e.g., approximately 680 nanometers (approximately 440 terahertz).

Second received electromagnetic radiation 1104 preferably is used to compensate first received electromagnetic radiation 1004 for the effects of moisture content variation of the epidermis E. Preferably, second received electromagnetic radiation 1104 is a measure of the moisture content of the epidermis E and is used to compensate first received electromagnetic radiation 1004 in order to mitigate the effect of moisture content variation of the epidermis E, which is the source of the problem that the inventors discovered. Accordingly, the reliability of measurements made with bi-spectral sensor 1100 for aiding in diagnosing if an infiltration/extravasation event has occurred preferably is improved.

FIG. 4B shows system 1100 preferably includes a processor 1120 running an algorithm that mitigates the effect of epidermal moisture content variations when aiding in diagnosing if an infiltration/extravasation event has occurred. Preferably, processor 1120 receives first received electromagnetic radiation 1004 and second received electromagnetic radiation 1104. Processor 1120 preferably analyzes second received electromagnetic radiation 1104 to determine if there is epidermal moisture content variation and, if so, whether it is decreasing from a generally heightened level or increasing from a generally depressed level. Preferably, processor 1120 compensates first received electromagnetic radiation 1004 as necessary based on the analysis of second received electromagnetic radiation 1104. Accordingly, changes in first received electromagnetic radiation 1004 that are due to moisture content variations of the epidermis E, which is the source of the problem that the inventors discovered, preferably may be mitigated to make system 1100 more reliable for aiding in diagnosing if an infiltration/extravasation event has occurred.

FIG. 5A shows a system 1200 including a combination sensor 1210 coupled with the epidermis E for (i) aiding in diagnosing infiltration or extravasation; and (ii) measuring moisture content variation of the epidermis E. Aiding in diagnosing infiltration or extravasation preferably includes electromagnetic radiation 1002 that is emitted via a sensor face 1210a of combination sensor 1210 and electromagnetic radiation 1004 that is received via sensor face 1210a. Preferably, emitted and received electromagnetic radiations 1002 and 1004 of combination sensor 1210 and of electromagnetic spectrum sensor 1000 (FIG. 1) are generally similar. For example, emitted and received electromagnetic radiations 1002 and 1004 of both sensors preferably include percutaneous signals in the near-infrared portion of the electromagnetic spectrum.

Measuring epidermal moisture content with combination sensor 1210 preferably includes measuring at least one cutaneous property that correlates with the moisture content of the epidermis E. Preferably, combination sensor 1210 includes a probe 1212 for measuring an electrical property of the epidermis E. According to one embodiment, probe 1212 preferably includes an anode 1212a and a cathode 1212b that contiguously engage individual points of the epidermis E. Preferably, anode 1212a and cathode 1212b measure resistance, impedance, capacitance, inductance or another electrical property of the epidermis E that correlates with the moisture content of the epidermis E. According to other embodiments, probe 1212 may measure a change in an electrical or magnetic field that correlates with variations in the moisture content of the epidermis E.

The output of probe 1212 preferably is used to compensate received electromagnetic radiation 1004 for the effects of moisture content variation of the epidermis E. Preferably, measurements by probe 1212 correlate with moisture content variations of the epidermis E and the output of probe 1212 is used to compensate received electromagnetic radiation 1004 in order to mitigate the effect of moisture content variation of the epidermis E, which is the source of the problem that the inventors discovered. Accordingly, the reliability of measurements made with combination sensor 1210 for aiding in diagnosing if an infiltration/extravasation event has occurred preferably is improved.

FIG. 5B shows system 1200 preferably includes a processor 1220 running an algorithm that mitigates the effect of epidermal moisture content variations when aiding in diagnosing if an infiltration/extravasation event has occurred. Preferably, processor 1220 receives received electromagnetic radiation 1004 and the output of probe 1212. Processor 1220 preferably analyzes the output of probe 1212 to determine if there is epidermal moisture content variation and, if so, whether it is decreasing from a generally heightened level or increasing from a generally depressed level. Preferably, processor 1220 compensates received electromagnetic radiation 1004 as necessary based on the analysis of the output of probe 1212. Accordingly, changes in received electromagnetic radiation 1004 that are due to moisture content variations of the epidermis E, which is the source of the problem that the inventors discovered, preferably may be mitigated to make system 1200 more reliable for aiding in diagnosing if an infiltration/extravasation event has occurred.

While the present invention has been disclosed with reference to certain embodiments, numerous modifications, alterations and changes to the described embodiments are possible without departing from the sphere and scope of the present invention, as defined in the appended claims. For example, emitted and received electromagnetic radiations 1002 and 1004 may be centered about a peak wavelength at which the effect of cutaneous moisture may be generally insignificant as compared to subcutaneous fluid. According to one embodiment, electromagnetic radiation preferably is centered about a peak wavelength that is readily absorbed by a common constituent in both sweat and infusates. Water, which is an example of such a constituent, is readily absorbed in a band of wavelengths from approximately 940 nanometers to approximately 970 nanometers (approximately 319 terahertz to approximately 309 terahertz). With regard to received electromagnetic radiation 1004 in this band, changes due to sweat may be insignificant as compared to changes due to infiltration/extravasation because the volume of moisture at the epidermis E may be relatively small as compared to the volume of fluid in the perivascular tissue P. Accordingly, an aid to indicate an infiltration/extravasation event preferably is based on changes in electromagnetic radiation 1004 that exceed a selected threshold such that changes in electromagnetic radiation 1004 below the selected threshold may be disregarded as being due to, for example, epidermal moisture content variations.

According to another example, the intensity of emitted electromagnetic radiation 1002 may be selected or varied so as to render the effect of cutaneous moisture generally insignificant as compared to the effect of subcutaneous fluid. Based again on water being a common constituent in both infusates and sweat, a relatively larger volume of water in the perivascular tissue P during an infiltration/extravasation event generally has a greater limit to absorb emitted electromagnetic radiation 1002 as compared to a relatively smaller volume of water at the epidermis E due to sweat. Preferably, the intensity of emitted electromagnetic radiation 1002 is selected to be greater than that which can be absorbed by epidermal moisture and less than that which can be absorbed by perivascular fluid. Accordingly, received electromagnetic radiation 1004 preferably is sensitive to subcutaneous fluid changes as an aid to indicate an infiltration/extravasation event and relatively insensitive to cutaneous moisture variations, which are generally less significant because emitted electromagnetic radiation 1002 saturates epidermal moisture, e.g., sweat.

According to a further example, electromagnetic spectrum sensor 1000 preferably differentiates between the spectral signatures of sweat and infusates as an aid in diagnosing an infiltration/extravasation event. Preferably, distinguishing between the spectral signatures of subcutaneous fluid and cutaneous moisture facilitates mitigating the effect of epidermal moisture content variations on received electromagnetic radiation 1004. Accordingly, electromagnetic spectrum sensor 1000 preferably provides an aid to indicate an infiltration/extravasation event has occurred based on detecting the spectral signature of the infusate in the perivascular tissue P.

While the present invention has been disclosed with reference to annulling or compensating for variations in moisture content of the epidermis to make percutaneous electromagnetic radiation measurements reliable, other mitigating systems and methods are possible to aid in diagnosing subcutaneous fluid leakage, monitoring an intravascular infusion, or monitoring over time changes in an anatomical property. For example, extenuating or palliating variations in moisture content of the epidermis may also make percutaneous electromagnetic radiation measurements more reliable. Thus, It is intended that the present invention not be limited to the described embodiments, but that it has the full scope defined by the language of the following claims, and equivalents thereof.

What is claimed is:

1. A system for monitoring an intravascular infusion, the apparatus comprising:
a sensor configured to emit a first percutaneous electromagnetic signal and to receive a second percutaneous electromagnetic signal, the second percutaneous electromagnetic signal including at least one of a reflection, scattering and diffusion of the first percutaneous electromagnetic signal; and
antiperspirant configured to be disposed at a target area of epidermis, wherein the first and second percutaneous electromagnetic signals pass through the target area.

2. The system of claim 1 wherein the first and second percutaneous electromagnetic signals comprise near-infrared signals.

3. The system of claim 1 wherein the first percutaneous electromagnetic signal is configured to impinge on perivascular tissue, and wherein the second percutaneous electromagnetic signal includes at least one of reflection, scattering and diffusion of the first percutaneous electromagnetic signal by the perivascular tissue.

4. The system of claim 1, comprising a barrier film configured to coat the target area with a protective layer.

5. The system of claim 4 wherein the barrier film consists of at least one of Cavilon™ and Skin-Prep™.

6. A system for monitoring a subcutaneous anatomical property of a body that includes an epidermis, the system comprising:
a first sensor configured to overlie a target area of the epidermis and to receive a first signal regarding the subcutaneous anatomical property; and
a second sensor configured to receive a second signal regarding moisture content of the target area of the epidermis.

7. The system of claim 6 wherein the first sensor comprises an aid to diagnosing at least one of infiltration and extravasation.

8. The system of claim 6 wherein the first signal comprises a near-infrared electromagnetic signal.

9. The system of claim 6 wherein the second signal comprises a visible light electromagnetic signal.

10. The system of claim 6 wherein the second sensor comprises at least one of an epidermal resistance sensor, an epidermal impedance sensor, and an epidermal temperature sensor.

11. The system of claim 6 wherein the second sensor comprises at least one of a magnetic field sensor and an electrical field sensor.

12. The system of claim 6, comprising a processor including an algorithm receiving the first and second signals, wherein the algorithm compensates the first signal for variations in the moisture content of the target area based on the second signal.

13. A method of sensing fluid in perivascular tissue, the method comprising:
applying an antiperspirant to a target area of epidermis; and
coupling a sensor to the epidermis, the sensor being configured to emit and detect near-infrared signals through the target area.

14. The method of claim 13 wherein applying the antiperspirant comprises distributing on the target area a layer consisting of at least one of aluminum chloride, aluminum zirconium, aluminum chlorohydrate, aluminum hydroxybromide, crystal alum and talc.

15. The method of claim 13 wherein the sensor includes a foundation and coupling the sensor comprises adhering the foundation to the epidermis.

16. The method of claim 15 wherein the foundation comprises a medical grade adhesive that is biocompatible according to at least one of ISO 10993 and USP Class VI.

17. The method of claim 16 wherein the foundation comprises a panel that is generally transparent to the near-infrared signals.

18. The method of claim 13 wherein coupling the sensor comprises overlaying a dressing on the epidermis and inserting the sensor in the dressing over the target area.

19. The method of claim 13, comprising cleaning the target area with an antiseptic.

20. The method of claim 13, comprising protecting the epidermis with a barrier film.
